(19) United States
(12) Patent Application Publication
(10) Pub. No.: US 2010/0291606 A1
(43) Pub. Date: Nov. 18, 2010

(54) SCREENING SYSTEM FOR DETECTING INHIBITORS OF HIV INTEGRASE-LEDGF/p-75 INTERACTION

(75) Inventors: Manuel Llano, El Paso, TX (US); Elisa Morales, El Paso, TX (US)

Correspondence Address: CHALKER FLORES, LLP, 2711 LBJ FRWY, Suite 1036, DALLAS, TX, 75234, US

(73) Assignee: BOARD OF REGENTS, THE UNIVERSITY OF TEXAS SYSTEM, Austin, TX (US)

(21) Appl. No.: 12/779,154

(22) Filed: May 13, 2010

Related U.S. Application Data
(60) Provisional application No. 61/177,817, filed on May 13, 2009.

(51) Int. Cl. C12Q 1/48

(52) U.S. Cl. 435/15

(57) ABSTRACT

The development and validation of a cell-based, homogeneous high throughput screening (HTS) assay for small compounds inhibiting the HIV integrase-LEDGF/p75 interaction is described herein. The HTS strategy has the potential to identify small-molecules interfering with the interaction of HIV integrase-LEDGF/p75. These small molecules represent starting scaffolds for therapeutic drug development.

### CROSS-REFERENCE TO RELATED APPLICATIONS

This application claims priority to U.S. Provisional Application Ser. No. 61/177,817, filed May 13, 2009, the entire contents of which is incorporated herein by reference.

### STATEMENT OF FEDERALLY FUNDED RESEARCH

This invention was made with U.S. Government support under Contract Nos. 1SC2GM082301-01 and 5G12RR008124 awarded by the NIH. The government has certain rights in this invention.

### TECHNICAL FIELD OF THE INVENTION

The present invention relates in general to the field of development and detection of compounds inhibiting the HIV viral life cycle, and more particularly to development and validation of a high throughput screening HTS assay for small compounds inhibiting the HIV integrase-LEDGF/p75 interaction.

### INCORPORATION-BY-REFERENCE OF MATERIALS FILED ON COMPACT DISC

None.

### BACKGROUND OF THE INVENTION

Without limiting the scope of the invention, its background is described in connection with the HIV viral life cycle and more specifically to the identification of compounds interfering with HIV integrase.

United States Patent Application 20070259358 (Debyser, et al., 2007) discloses a cellular protein that associates with integrase (integrase interacting protein-Inip), to molecules interacting with Inip and their use as an antiviral. The invention also relates to antibodies, RNA interference, antigen therapy, gene silencing or antisense inhibition of said integrase interacting protein. The novel integrase interaction protein is a target for HIV replication prevention or inhibition.

WIPO Patent Application No. WO/2008/068765 (Friedler, et al., 2008) provides isolated peptides comprising a fragment of an HIV-1 Rev protein used for the treatment of HIV-1 infection, inhibiting HIV-1 replication and inhibiting DNA binding and 3′-end processing activity of HIV-1 integrase protein.

Christ and colleagues have developed a high-throughput assay to discover inhibitors of the interaction of HIV integrase with the host protein LEDGF/p75 and found compounds with low micromolar activity. They authors consider this interaction a viable target for drug development. By solving the X-ray structure of IN-core complexed with the integrase binding domain (IBD) of p75 showing a well-defined interphase. The authors suggest that peptides based on the p75 “KID”-loop would be effective inhibitors of the interaction. Likewise the apparent stability of the p75 surface is promising for binding IN-derived peptides. Both strategies were applied in designing libraries of peptides targeting the IN-p75 interaction. Essential amino acid residues of the most active peptides were projected on natural occurring stable helical folds in order to improve inhibitory activities. For in vitro hit identification the authors developed a high throughput Alphascreen assay monitoring protein protein interaction. JPO2 has emerged as the predominant physiological binding partner of LEDGF/p75 in cells. To predict potential cellular toxicity the authors established a counterscreen to further evaluate identified hits. Antiviral activity and cytotoxicity of initial hits from in vitro screening were evaluated in cell culture by MTT/MT4. Purification of recombinant IN, LEDGF/p75, and JPO2 was optimized in order to set up a high-throughput screening assay for hit identification. Thorough evaluation of the LEDGF/p75-IN and LEDGF/p75-JPO2 Alphascreen assays evidences its applicability for antiviral drug discovery. By rational peptide design the authors generated potent inhibitors of the LEDGF/p75-IN interaction with IC₅₀ in the low to sub-micromolar range, with the strongest inhibition observed for PEA-64 (IC₅₀=0.35 μM). In cell culture the identified hits, fused to a transduction domain, demonstrate moderate antiviral activity at non-toxic concentrations. The identification of the first inhibitors of LEDGF/p75-IN interaction validated this interaction as a target for antiviral therapy.

### SUMMARY OF THE INVENTION

The present invention describes a high-throughput screening assay for small molecule inhibitors of inhibitors of the HIV integrase-LEDGF/p75 interaction. In one embodiment the present invention is a screening method for one or more small molecule inhibitors of HIV-1 integrase. The method of the present invention involved providing one or more engineered cells expressing the HIV-1 integrase, wherein the expressed HIV-1 integrase comprises an attached green fluorescent protein. A baseline green fluorescence signal emanating from the green fluorescent protein attached to the HIV-1 integrase is measured followed by the addition of the one or more small molecule inhibitors dissolved or dispersed in an aqueous or an organic solvent to the one or more engineered cells. Finally, a second test green fluorescence signal resulting from the interaction of the one or more small molecule inhibitors and the expressed HIV-1 integrase is measured.

In one aspect of the present invention the method further comprises the step of measuring a cherry fluorescence signal at periodic intervals. The cherry fluorescent signal emanates from a cherry fluorescent protein contained in the one or more engineered cells and is indicative of a health of the one or more engineered cells.

In another aspect the one or more engineered cells comprises at least two fluorescent proteins. In a further aspect the one or more engineered cells expresses at least one fluorescent protein attached to the expressed HIV-1 integrase. In yet another aspect the one or more engineered cells expresses at least one fluorescent protein that is not attached to the HIV-1 integrase.

One aspect of the present invention is directed towards the one or more small molecule inhibitors that are selected from a group comprising of organic compounds, heterocyclic aromatic and acyclic heteroatom compounds, dioxybutyric acids and derivatives, propanediones and derivativess, naphthyridine-carboxamides and derivatives, naphthalenyl ketones and derivativess, hydroxynaphthyridinone carboxamides and derivatives, hydroxypyrrole derivatives, tricyclic analogs of hydroxy polyhydronaphthyridine dione compounds and nitrogenous condensed ring compounds. The one or more small molecule inhibitors are dissolved in an aqueous solvent of an organic solvents comprising of ketones, alcohols, dimethyl sulfoxide, esters, ethers, acids or any combinations thereof.

In a certain aspect the one or more engineered cells are selected from cells, cell lines, or cell cultures selected from a group comprising of human embryonic kidney cells, Chinese hamster ovary cells, HeLa cell lines, COS cell lines, mammalian cells and bacterial cells.

In related aspects a decrease in intensity of the test green fluorescent signal when compared to the intensity of the baseline green fluorescent signal indicates inhibition of the one or more enzyme by the one or more small molecule inhibitors and a decrease in the intensity of the cherry fluorescent signal over time indicates deteriorating health of the one or more engineered cells or the cell line or the cell culture.

In another embodiment of the present invention describes a method for screening one or more small molecule inhibitors of HIV-1 integrase. The method comprises of providing one or more engineered human embryonic kidney cells expressing the HIV-1 integrase, wherein the expressed HIV-1 integrase comprises an attached green fluorescent protein, this is followed measuring a baseline green fluorescence signal emanating from the green fluorescent protein attached to the HIV-1 integrase. The one or more small molecule inhibitors dissolved or dispersed in an aqueous or an organic solvent are added to the one or more engineered human embryonic kidney cells and finally a second test green fluorescence signal resulting from the interaction of the one or more small molecule inhibitors and the expressed HIV-1 integrase is measured.

In a further aspect the method comprises the step of measuring a cherry fluorescence emanating from a cherry fluorescent protein contained in the one or more engineered human embryonic kidney cells at periodic intervals, wherein the cherry fluorescence is indicative of a health of the one or more engineered human embryonic kidney cells.

In one aspect the one or more small molecule inhibitors are selected from a group comprising of organic compounds, heterocyclic aromatic and acyclic heteroatom compounds, dioxybutyric acids and derivatives, propanediones and derivativess, naphthyridine-carboxamides and derivatives, naphthalenyl ketones and derivativess, hydroxynaphthyridinone carboxamides and derivatives, hydroxypyrrole derivatives, tricyclic analogs of hydroxy polyhydronaphthyridine dione compounds and nitrogenous condensed ring compounds.

In another aspect of the present invention a decrease in intensity of the test green fluorescent signal when compared to the intensity of the baseline green fluorescent signal indicates inhibition of the HIV-1 integrase enzyme by the one or more small molecule inhibitors. In yet another aspect of the present invention a decrease in the intensity of the cherry fluorescent signal over time indicates deteriorating health of the one or more engineered human embryonic kidney cells.

### BRIEF DESCRIPTION OF THE DRAWINGS

For a more complete understanding of the features and advantages of the present invention, reference is now made to the detailed description of the invention along with the accompanying figures and in which:

FIG. 1 is a schematic representation of the LEDGF/p75 protein modular structure. The chromatin-binding domain is formed by the functional interaction of the PWWP domain and two AT hooks motifs. The integrase binding domain mediates the interaction of LEDGF/p75 with HIV integrase and several cellular proteins;

FIG. 2 is a schematic showing the LEDGF/p75 tethering model. Based in the study of the HIV co-factor activity of LEDGF/p75 mutants it is postulated that chromatin bound LEDGF/p75 tethers integrase linked to the pre-integration complex to the host chromatin facilitating viral integration;

FIG. 3 is a schematic showing the role of LEDGF/p75 in protecting HIV-1 integrase from proteasomal-mediated degradation;

FIG. 4 is the HIV IN-eGFP expression plasmid used to develop the IN-eGFP cell line. The expression of IN-eGFP is driven by the CMV immediate early promoter and linked through an internal ribosome entry site (IRES) to the puromycin N-acetyltransferase (PAC) gen;

FIG. 5 shows the results obtained from the FACS analysis of IN-eGFP cells. High levels of green fluorescence were detected in cells stably transfected with the expression plasmid encoding IN-eGFP-IRES-PAC (FIG. 5). Parental HEK293T cells were used as a negative control;

FIG. 6 shows the images from the confocal microscopy analysis of IN-eGFP cells. IN-eGFP was exclusively localized to the nuclear compartment of these cells;

FIG. 7 shows the immunoblot images of LEDGF/p75 levels in 2L_{KD}-IN-eGFP cells. LEDGF/p75 levels were analyzed by immunoblotting in single cell clones transduced with a lentivirus expressing a shRNA specific to LEDGF/p75 or a control shRNA were analyzed. GAPDH was determined in the same immunoblot membrane as a loading control;

FIG. 8 shows the results of the FACS analysis of IN-eGFP- and 2L_{KD}-IN-eGFP-derived single cell clones. Green fluorescence levels were routinely higher in IN-eGFP-derived cell clones (LEDGF/p75+) than in 2L_{KD}-IN-eGFP-derived cell clones (LEDGF/p75−);

FIG. 9 shows that sub-cellular distribution of IN-eGFP in IN-eGFPc and 2L_{KD}-IN-eGFP cell lines. The nuclear localization of IN-eGFP in the LEDGF/p75+ cells IN-eGFP cells was lost after efficient knockdown of LEDGF/p75 (2L_{KD}-IN-eGFP cells);

FIGS. 10A-10C shows the confocal microscopy analysis results indicating that wild type LEDGF/p75 (FIG. 10B), but not the ΔIBD mutant (FIG. 10C), can rescue nuclear and chromosome localization of IN-eGFP in 2L_{KD}-IN-eGFP cells;

FIG. 11 shows the FACS analysis results indicating that wild type LEDGF/p75, but not the ΔIBD mutant, can increase the green fluorescence levels of 2L_{KD}-IN-eGFP cells. Levels of re-expressed LEDGF proteins were verified by immunoblotting; and

FIG. 12 shows the fluorescence microscopy images indicating that Wild type LEDGF/p75, but not the ΔIBD mutant, can increase the green fluorescence levels of 2L_{KD}-IN-eGFP cells.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein the “human immunodeficiency virus (HIV-1)” is used to describe the viral causative agent which is responsible for producing HIV infections in human patients, which, if untreated or unresolved will often result in AIDS or related immunological conditions and eventually death in a patient. The term “antiretroviral agent”, as used herein, includes any pharmacological, biological or cellular agent that has demonstrated the ability to inhibit HIV replication.

As used herein the terms “protein”, “polypeptide” or “peptide” refer to compounds comprising amino acids joined via peptide bonds and are used interchangeably. A “polypeptide” is a polymer of amino acid residues joined by peptide bonds, whether produced naturally or synthetically. Polypeptides of less than about 10 amino acid residues are commonly referred to as “peptides.” A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless. The term “green fluorescent protein” is simply historical as the original proteins, isolated from fluorescent organisms fluoresced in the green portion of the spectrum. In addition, Griffin, B. A., et al., Science (1998) 281:269-272, describe a technique for labeling individual cellular proteins with fluorescent probes and tracing their intracellular location.

The term “enzyme” encompasses a large number of protein biological catalysts, which are known to or are predicted to catalyze a reaction. Most commonly, an enzyme can catalyze at least one of many different possible biochemical reactions that comprise biological pathways. Further, an enzyme can catalyze an organic chemical reaction, such as conversion of ethanol to acetic acid, or an inorganic reaction, such as reduction of molecular nitrogen. The terms “HIV integrase” and “integrase” as used herein are used interchangeably and refer to the integrase enzyme encoded by the human immunodeficiency virus type 1 or 2.

In the broadest sense the term “DNA” refers to deoxyribonucleic acids comprising bases selected from the group consisting of (1) naturally occurring modified or unmodified bases or (2) synthetically modified or unmodified bases. Naturally occurring bases can include, e.g., unmodified bases include thymidine, guanidine, adenine, cytosine and deoxyuridine. Naturally occurring or synthetically modified bases include, e.g., 4-acetylcytidine, 5-(carboxyhydroxylmethyl)uridine, 2′-methylcytidine, 5-carboxymethylaminomethyl-2-thiouridine, 5-carboxymethylaminomethyluridine,2′-O-methylpseudouridine, beta, D-galactosylqueosine, 2′-O-methylguanosine, inosine, N6-isopentenyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, beta, D-mannosylqueosine, 5-methoxycarbonylmethyluridine, 5-methoxyuridine, 2-methylthio-N6-isopentenyladenosine, N-((9-beta-D-ribofuranosyl-2-methylthiopurine-6-yl) carbamoyl) threonine, N-((9-beta-D-ribofuranosylpurine-6-yl)N-methylcarbamoyl) threonine, uridine-5-oxyacetic acid methylester, uridine-5-oxyacetic acid (v), wybutoxosine, pseudouridine, queosine, 2-thiocytidine, 5-methyl-2-thiouridine, 5-methyluridine, N-((9-beta-D-ribofuranosylpurine-6-yl)carbamoyl)threonine, 2′-O-methyl-5-methyluridine, 2′-O-methyluridine, wybutosine, (acp3)u. Additionally the term DNA includes all known types of DNA, e.g., RDNA, CDNA, genomic DNA, or ZDNA, as single stranded, double stranded or triple stranded, linear or circular. As used herein the term “mutation” indicates an alteration in the base sequence of a DNA strand compared to a reference strand.

The term “expression vector” or “vector” as used herein is a nucleic acid molecule encoding a gene that is expressed in a host cell. Typically, an expression vector comprises a transcription promoter, a gene, and a transcription terminator. Gene expression is usually placed under the control of a promoter, and such a gene is said to be “operably linked to” the promoter. Similarly, a regulatory element and a core promoter are operably linked if the regulatory element modulates the activity of the core promoter. The term “plasmid” as used herein includes any type of replication vector which has the capability of having a non-endogenous DNA fragment inserted into it. Procedures for the construction of plasmids include those described in Maniatis et al., Molecular Cloning, A Laboratory Manual, 2d, Cold Spring Harbor Laboratory Press (1989).

As used herein, the term “lentiviral vector” is intended to mean an infectious lentiviral particle. Lentivirinae or lentivirus, is a subfamily of enveloped retrovirinae or retroviruses, that are distinguishable from oncovirinae and spumavirinae based on virion structure, host range and pathological effects. For example, an infectious lentiviral particle will be capable of invading a target host cell, include an envelope and exhibit one or more characteristics of a lentivirus. Such characteristics include, for example, infecting non-dividing host cells, transducing non-dividing host cells, infecting or transducing host immune cells, containing a lentiviral virion including one or more of the gag structural polypeptides p7, p24 or p17, containing a lentiviral envelope including one or more of the env encoded glycoproteins p41, p120 or p160, containing a genome including one or more lentivirus cis-acting sequences functioning in replication, proviral integration or transcription, containing a genome encoding a lentiviral protease, reverse transcriptase or integrase, or containing a genome encoding regulatory activities such as Tat or Rev.

The term “genome” in its broadest sense is the genetic material of an organism. In some instances, the term genome may refer to the chromosomal DNA. Genome may be multichromosomal such that the DNA is cellularly distributed among a plurality of individual chromosomes. The term genome may also refer to genetic materials from organisms that do not have chromosomal structure. In addition, the term genome may refer to mitochondria DNA

As used herein, the term “polymerase chain reaction (PCR),” refers to the method of K. B. Mullis U.S. Pat. Nos. 4,683,195, 4,683,202, and 4,965,188, hereby incorporated by reference, which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”. With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of 32P-labeled deoxynucleotide triphosphates, such as DCTP or DATP, into the amplified segment). In addition to genomic DNA, any oligonucleotide sequence can be amplified with the appropriate set of primer molecules. In particular the amplified segments created by the PCR process itself are, themselves, efficient templates for subsequent PCR amplifications.

The term “high throughput screening” is a screening assay which is performed to test tens of or hundreds of samples simultaneously. For example, using a 16, 24, 48, 96, or 384 well plate to do a bioassay to test multiple samples simultaneously is considered “high throughput screening.”

The terms “immunoblot” and “Western blot” refer to methods of detecting a specific protein or proteins in a complex protein mixture such as a cell extract or lysate. These methods, which are well known in the art (See, e.g., Towbin et al, Proc Natl Acad Sci USA 76:4350-4354 [1979]; and Ausubel et al. (eds.), Current Protocols in Molecular Biology, John Wiley & Sons, Inc., New York [1994]), involve fractionating the protein mixture by SDS-polyacrylamide gel electrophoresis, transferring the separated proteins onto a solid support such as nitrocellulose and detecting the protein(s) of interest by with an antibody. The bound primary antibody can be visualized by the use of a secondary antibody conjugated to an enzyme which produces a signal in the presence of a suitable substrate.

The present invention describes a cellular-based screening system for the identification of small-molecules that disrupt the role of LEDGF/p75 in HIV infection. The invention further describes the development and validation of a high-throughput screening (HTS) strategy to identify small-molecules interfering with the interaction of HIV integrase—LEDGF/p75. These small-molecules represent starting scaffolds for therapeutic drug development.

In the last 23 years, the Human immunodeficiency virus (HIV) infection has reached pandemic levels with an estimate of 65 million infected people by 2006. Non-controlled HIV infection can result in the acquired immunodeficiency syndrome (AIDS) that has caused 25 million deaths by 2006. According to the Center of Disease Control, 1.1 million people are infected with HIV in the US. This number is predicted to considerable increase due to antiretroviral drug treatments continue to provide a larger expectancy of life to people with HIV.

HIV proteins are the main targets of the anti-retroviral treatment. The compounds used, block the HIV life cycle allowing the decrease of symptoms and suppression of the viral load. These compounds are classified in three categories: protease inhibitors, nucleoside reverse transcriptase inhibitors and non-nucleoside reverse transcriptase inhibitors. These inhibitors block the earlier step in viral infection and also arrest maturation of the virus and consequently block infection of vulnerable cells.

HIV has a high rate of mutation, thus currently available drugs targeting viral proteins frequently encounter drug-resistant viruses. A viable alternative to this limitation is to develop drugs that target cellular factors required for HIV infection but that are not essential to the cell. It is expected that the emergence of drug-resistant viruses will substantially decrease by using drugs that target these cellular co-factors. LEDGF/p75 is the first cellular factor to be identified that is required for HIV infection but is dispensable or normal cellular physiology [1].

HIV enters the target cell by fusion of the viral and cellular membranes mediated by the specific interaction of viral surface glycoproteins with cellular receptors. Immediately after entry, the viral RNA genome is reverse transcribed to a double-stranded DNA copy, which associates with viral and cellular proteins forming the preintegration complex (PIC). This complex is imported into the nucleus of the infected cell where the viral DNA is integrated into the host chromatin [2].

Integration of the viral DNA into the host chromatin is then catalyzed by the viral enzyme integrase (IN), which performs two enzymatic activities essential for viral integration; 3′ processing of the linear viral genome and DNA strand transfer of the viral genome into the host genome [2]. 3′ processing occurs soon after reverse transcription when a dinucleotide from each HIV-1 end is cleaved off, leaving two sticky ends on either end of the viral DNA [2]. IN's second enzymatic activity, DNA strand transfer, takes place after the PIC enters the nucleus and locates a suitable integration site. Then, IN cleaves the chromosomal DNA and integrates the viral genome. The resulting DNA recombination intermediate harbors single strand gaps that are then repaired by the host DNA repair machinery allowing for a complete provirus formation. It is only after integration, that proviral DNA is efficiently transcribed and the new viral progeny is generated, making integration an essential step in HIV infection [2].

The HIV integration process is not completely understood yet. It is though that integration requires the concerted action of both viral and cellular proteins [3,4]. Apparently, the integrase is the only viral protein essential for this process. Integrase interacts with the cellular protein LEDGF/p75 and this interaction promotes HIV-1 integration as well as influences the integration site distribution in the host chromatin [1,5,6].

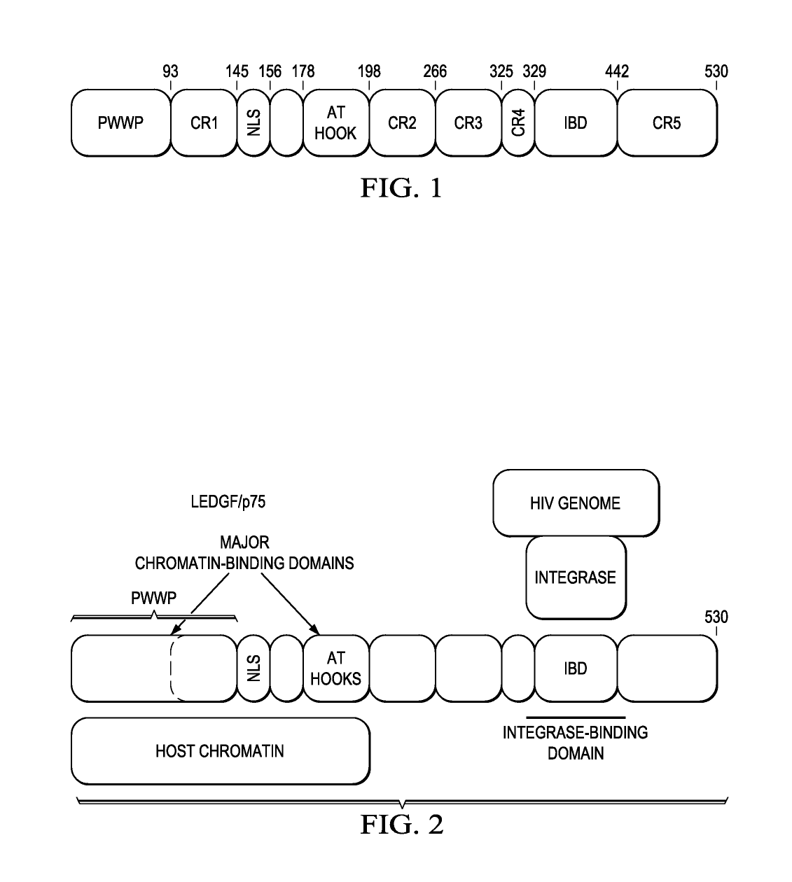

Role of LEDGF/p75 in HIV-1 infection: LEDGF/p75 is a ubiquitously expressed chromatin bound protein that belongs to the hepatoma-derived growth factor family. Chromatin binding is mediated by the functional interaction of the PWWP domain and two AT hooks motifs, all located in the N-terminus of this protein (FIG. 1) [7,8]. In the C-terminal region of LEDGF/p75, an evolutionarily conserved region called the integrase binding domain (IBD) allows the interaction of LEDGF/p75 with cellular proteins and with the viral integrase [9,10,11,12].

Cells severely depleted of LEDGF/p75 are resistant to HIV-1 infection because a defect in viral integration[1,6]. HIV-1 susceptibility is rescued in these cells upon re-expression of the LEDGF/p75 wild type protein. However, expression of LEDGF/p75 mutants lacking the chromatin binding capacity of the integrase binding domain did not rescue HIV-1 infectivity[1,6]. Based in these results, it was proposed that LEDGF/p75 acts as a molecular tether linking the HIV-1 integrase in the pre-integration complex to the host chromatin [1] (FIG. 2).

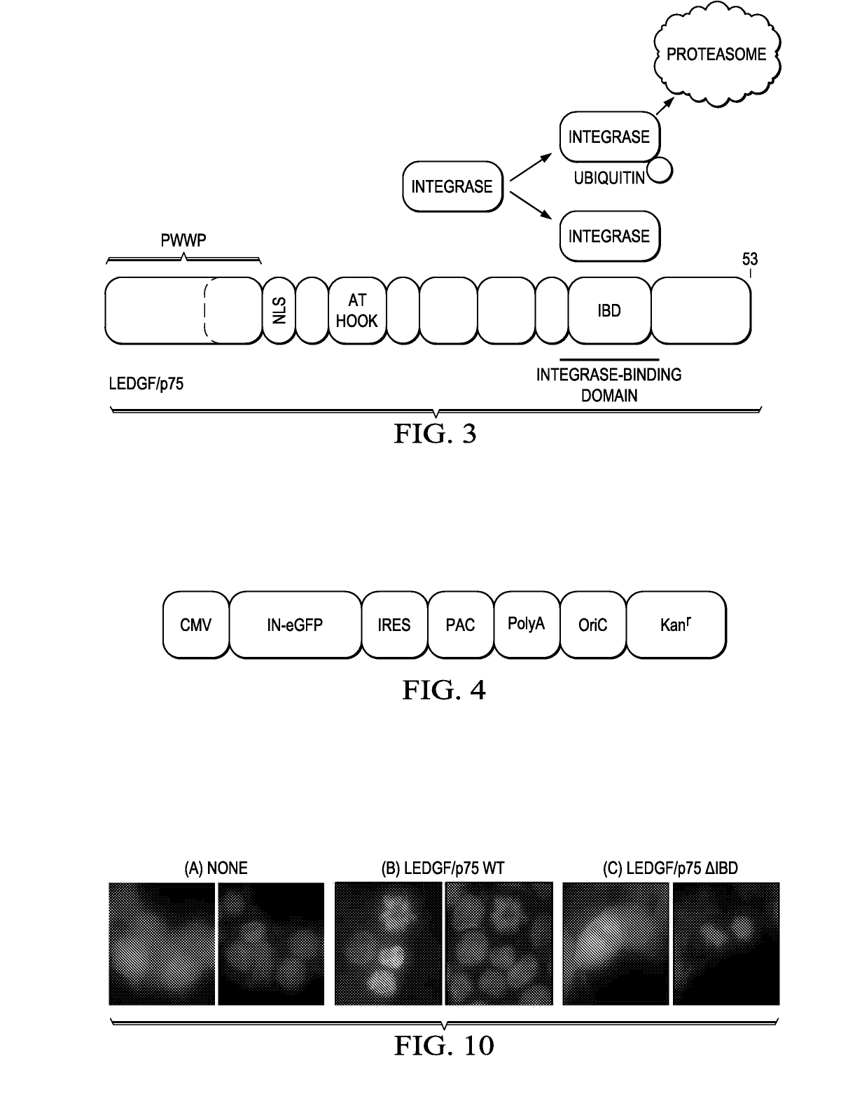

In addition to tethers integrase to the host chromatin, LEDGF/p75 protects the viral enzyme from proteasomal-mediated degradation [13]. The half-life of integrase in LEDGF/p75-deficient cells is importantly reduced; however, integrase mRNA levels are similar to the observed in control cells. Importantly, integrase degradation is prevented in these cells by re-expression of LEDGF/p75 wild type or by pharmacological inhibition of the proteasome [13]. LEDGF/p75 mutants lacking the IBD, however, did not protect integrase from proteasome degradation, indicating that this effect is dependent on the interaction of both proteins [13] (FIG. 3).

LEDGF/p75-Integrase interaction as an anti-HIV drug target: The interaction of LEDGF/p75 with HIV-1 integrase is a good candidate for anti-HIV drug development. LEDGF/p75, although essential for efficient HIV replication, is dispensable for the normal cellular physiology since cells lacking or severely deficient in LEDGF/p75 exhibited a normal phenotype in culture[1,6]. In addition, embryogenesis was not affected in a knockout mouse model, although musculoskeletal defects were observed in the progeny[14]. These results strongly suggest that important host toxicity is not expected with drugs affecting the interaction of LEDGF/p75 with HIV-1 integrase.

In addition to its role in HIV infection and in the cellular physiology, the structural bases of the LEDGF/p75-integrase interaction indicate that this is a druggable interphase. Their surface of interaction is relatively small compared to other protein-protein surface of interaction. Only three amino acids in LEDGF/p75 are required for integrase binding and the mutation of any of these residues abolishes this protein-protein interaction [15].

These results suggest that small-molecules could block the interaction of LEDGF/p75 with HIV-1 integrase and host toxicity is not expected with the use of these compounds.

HIV-1 infection has become a continuous challenge in the finding of therapeutic drugs to eradicate or at least decrease infection. The current anti-HIV therapy is based in the combination of drugs that target viral proteins involved in different steps of the viral life cycle. This treatment has substantially proven its effectiveness by extending the quality and length of life expectancy in HIV infected individuals. However, combination of multiple drugs is mandatory to reduce the development of drug-resistant viruses, a common outcome due to the high mutation rate of HIV proteins. Multi-drug therapies increase the cost and toxicity of the anti-HIV treatment, severely limiting its use worldwide and leading to the failure in preventing infection spreading.

A second generation of anti-HIV drugs targeting cellular cofactors dispensable for cells but required for HIV infection, such as LEDGF/p75, is currently in development. These drugs are intended to block the interaction of the cellular cofactor with its viral counterpart affecting HIV-1 infection. Due to the low rate of mutation of cellular proteins, it is unlikely the development of drug resistance mechanisms affecting the second generation of anti-HIV drugs. Therefore, mono-drug regimes could be suitable alternatives for these drugs.

The present invention discloses the development and validation of a HTS for small compounds inhibitory of the HIV integrase-LEDGF/p75 interaction. The screening system is based in the ability of LEDGF/p75 to tether integrase to chromatin [16] and protect the viral enzyme from proteosomal-mediated degradation [13]. The inventors generated a HEK293T stable cell line expressing eGFP-tagged HIV integrase and monomeric Cherry, mCherry, (referred here as reporter cell line or IN-eGFPc cells). The inventors indicate that the drugs inhibiting the LEDGF/p75-integrase interaction will cause a redistribution of IN-eGFP and a protesome-mediated degradation of this fusion protein. These effects will be registered by measuring the green fluorescence levels and the subcellular distribution of IN-eGFP in the treated cells.

The inventors further validated the reporter cell line, by depleting the LEDGF/p75 in these cells by stable expression of a LEDGF/p75 specific shRNA. It is expected that fluorescence levels will be significantly reduced in knockdown cells and the nuclear localization of IN-eGFP will change. In addition, re-expression of LEDFG/p75 wild type will rescue fluorescence levels and nuclear localization of IN-eGFP in the LEDGF/p75-deficient cells.

The present disclosure describes the generation of a stable cell line expressing integrase-eGFP fusion protein and mCherry (IN-eGFPc cells) by:

a) Construction of a mammalian expression plasmid for the expression of HIV integrase-eGFP fusion protein.

b) Generation of stable cell lines expressing HIV IN-eGFP fusion protein and mCherry fluorescent protein in HEK293T cells.

c) Characterization of IN-eGFPc cells by FACS, confocal microscopy, and immunoblotting analyses.

In addition the present invention also describes the validation of IN-eGFP cells as a reporter system for drugs blocking the LEDGF/p75-IN interaction. In order to do this, LEDGF/p75 was depleted from IN-eGFP cells by shRNA expression using lentiviruses. (2L_{KD}-IN-eGFP cells).

a) Production of a lentiviral vector expressing a shRNA against LEDGF/p75.

b) Generation of LEDGF/p75-deficient cells in IN-eGFP cells.

c) Rescue of integrase expression in 2L_{KD}-IN-eGFP cells by re-expression of LEDGF/p75.

Generation of a stable cell line expressing integrase-eGFP fusion protein (IN-eGFP cells): A reporter cell line stably expressing integrase fused to eGFP was generated. This cell line allows visualizing in real time the subcellular distribution and levels of integrase proteins in cells. Drugs interfering with the LEDGF/p75-HIV integrase interaction are expected to trigger proteasome-mediated degradation of integrase and nuclear exclusion of integrase determining a decrease in fluorescence levels and a pancellular distribution of the fusion protein [13,16].

Construction of a mammalian expression plasmid for HIV integrase-eGFP fusion protein: The HIV-1 integrase eGFP expression plasmid was constructed by cloning an internal ribosome entry site (IRES)-puromycin N-acetyltransferase (pac) expression cassette into a unique BglII site in pHINeGFP (FIG. 4) [16]. IRES pac was PCR amplified from the pEFIRESp plasmid. Correct sequence of the construct was verified by DNA sequencing.

Generation of stable cell lines expressing HIV IN-eGFP fusion protein in HEK293T cells: HEK293T cells were used for the stable expression of HIVIN-eGFP-IRES-pac. The new constructed plasmid was transfected by the calcium phosphate method as described in [16]. Briefly, HEK293T cells were plated at 3×10⁶ cells in a T75 flask in 12 ml of culture medium. The expression plasmid was linearized at the prokaryotic backbone with a restriction enzyme. Transfection media with 20 ug of linearized DNA was added to the cells and incubated at 37° C. in the presence of 5% CO₂ and 95% humidity. After 24 hrs, culture media was changed for fresh media with 3 ug/ml puromycin antibiotic. Cells were grown in this selection medium until a resistant culture emerges.

Expression of mCherry: IN-eGFP cells were transduced with a lentiviral vector expressing mCherry, cells were single-cell cloned by limiting dilution and a clone expressing high levels of mCherry and IN-eGFP was selected for further characterization. These cells are referred in this disclosure as IN-eGFPc.

Characterization of IN-eGFPc cells by FACS, confocal microscopy, and immunoblotting analyses: Expression of IN-eGFP was evaluated in the puromycin resistant cells by fluorescence activated cell sorting (FACS) analysis. In addition, the correct size of the fusion protein was verified by immunoblotting with an anti-eGFP monoclonal antibody (Mab). Finally, subcellular distribution of IN-eGFP was determined by confocal microscopy analysis. mCherry levels were evaluated by confocal microscopy analysis.

For FACS analysis cells were harvested by trypsin treatment, washed in PBS and samples analyzed in the flow cytometer of the Cell Culture and High throughput Screening Core Facility. As a negative control, parental HEK293T cells were used.

For immunoblotting analysis procedures described in [16] were followed. Briefly, 10⁶ IN-eGFPc cells were lysed in 300 ul of RIPA buffer (150 mM Tris-HCl, pH 8.0, 150 mM NaCl, 0.5% DOC, 0.1% SDS, 1% NP-40) supplemented with protease inhibitors (final concentration: leupeptin 2 ug/ml, aprotinin 5 ug/ul, PMSF1 mM, pepstatin A 1 ug/ml) and centrifuged at 22, 000 g for five mins at 4° C. Cellular lysates, 10 ul, were resolved by SDS-PAGE and transfer overnight to PDVF membranes at 100 mAmp at 4° C. Membrane were blocked in TBS containing 10% milk for one hour and then incubated with primary antibodies diluted in TBS-5% milk-0.05% Tween-20 (antibody dilution buffer). IN-eGFP was detected with anti-eGFP monoclonal antibody (Mab) diluted 1/4000. As a loading control, anti-GAPDH Mab was used ( 1/2000). Membranes were incubated for two hrs at room temperature with, anti-eGFP, and anti-GAPDH Mabs and then membranes were washed three times for five minutes in TBS-0.1% Tween-20 (washing buffer). Bound antibodies were detected with goat anti-mouse Igs-HRP diluted 1/2000 in antibody dilution buffer followed by chemoluminescence detection.

For confocal microscopy analysis procedures described in [16] were followed. Briefly, cells were plated at 0.2×10⁶ cells in LabTek II chambered coverglasses. After 24 hrs incubation for attachment, cells were washed 3× with PBS, fixed with 4% formaldehyde in PBS for 10 mins at 37° C. then washed once with PBS and stained with DAPI. Then, cells were analyzed for subcellular distribution of HIV-1 IN-eGFP under the confocal microscope.

Development of a LEDGF/p75 deficient cell line in IN-eGFP cells (2L_{KD}-IN-eGFP cells): Drugs interfering with the LEDGF/p75-integrase interaction will change the subcellular localization of IN-eGFP as well as induce its degradation by the proteasome [13,16]. The inventors depleted the cellular LEDGF/p75 from IN-eGFP cells and FACS and confocal microscopy analysis, respectively, were used to study the levels of IN-eGFP and its subcellular distribution. Depletion of LEDGF/p75 mimics the lack of LEDGF/p75 available for interaction encountered in the presence of a drug that interferes with the interaction of this protein with integrase.

Production of a lentiviral vector expressing a shRNA against LEDGF/p75: LEDGF/p75 depletion of IN-eGFP was achieved by lentiviral transduction of a shRNA specific against LEDGF/p75. As control IN-eGFP cells were also transduced with a lentiviral vector expressing an scrambled shRNA sequence. These lentiviral vectors integrate into the host genome a cassette containing in cis an U6 small nuclear RNA promoter-driven shRNA expression cassette and a CMV-driven mCherry fluorescent protein expression cassette. This expression system allows selection of LEDGF/p75 knockdown cells based on their mCherry fluorescence levels [1].

Procedures described in [1] were followed for the production of the retroviral vectors. Briefly, HIV-derived vectors expressing anti-LEDGF/p75 shRNA were produced by calcium-phosphate co-transfection of HEK293T with 15 ug of pTSINcherry (LEDGF shRNA or control shRNA), 15 ug of pCMVΔR8.91 and 5 ug of the Vesicular Stomatitis Virus glycoprotein G (VSV-G) expression plasmid, pMD.G. Forty-eight hours after transfection, viral supernatants were harvested and concentrated by ultracentrifugation at 124,750 g for two hours on a 20% sucrose cushion. Concentrated vectors were used for transduction of IN-eGFP cells.

Generation of LEDGF/p75-deficient cells in IN-eGFP cells (2LKD-IN-eGFP cells) and control IN-eGFP cells (this cells are referred to in this disclosure as IN-eGFPc): IN-eGFP cells were transduced with the lentiviral vector expressing anti-LEDGF/p75 shRNA or control scrambled shRNA at a multiplicity of infection 300, as described before [1]. Twenty-four hrs later, the input viral vector was washed and cells were single-cell cloned by limiting dilution cultures. Clones expressing the highest levels of red fluorescence were selected for further analysis. Levels of cherry fluorescence were determined under the fluorescence microscope. Knockdown levels of LEDGF/p75 were verified further by immunoblotting with an anti-LEDGF Mab.

Rescue of integrase expression and nuclear localization in 2L_{KD}-IN-eGFP cells: The eGFP fluorescence levels of IN-eGFP cells decreased and nuclear localization of this fusion protein was altered following LEDGF/p75 depletion. In order to validate further that the change in levels and subcellular distribution of IN-eGFP are due to the lack of LEDGF/p75 and subsequently proteasome-mediated degradation of HIV integrase, rescue experiments were performed. Re-expression of LEDGF/p75 caused a re-bound in the green fluorescence levels of 2L_{KD}-IN-eGFP cells associated with a nuclear re-localization of IN-eGFP.

4×10⁵ 2L_{KD}-IN-eGFP cells were transfected with 2 ug of the expression plasmid pLEDGF/p75 WT-flag using the calcium phosphate method. Transfection medium was removed the next day and cells were analyzed twenty-four hrs later for green fluorescence levels by FACS and for subcellular distribution of IN-eGFP by fluorescence microscopy. Additionally, integrase levels were also determined by immunoblotting with anti-eGFP Mab. As control, a LEDGF/p75 mutant lacking the IBD was expressed in 2L_{KD}-IN-eGFP cells. Because this mutant does not interact with integrase, it is expected that the levels of green fluorescence will not increase in the transfected 2L_{KD}-IN-eGFP cells.

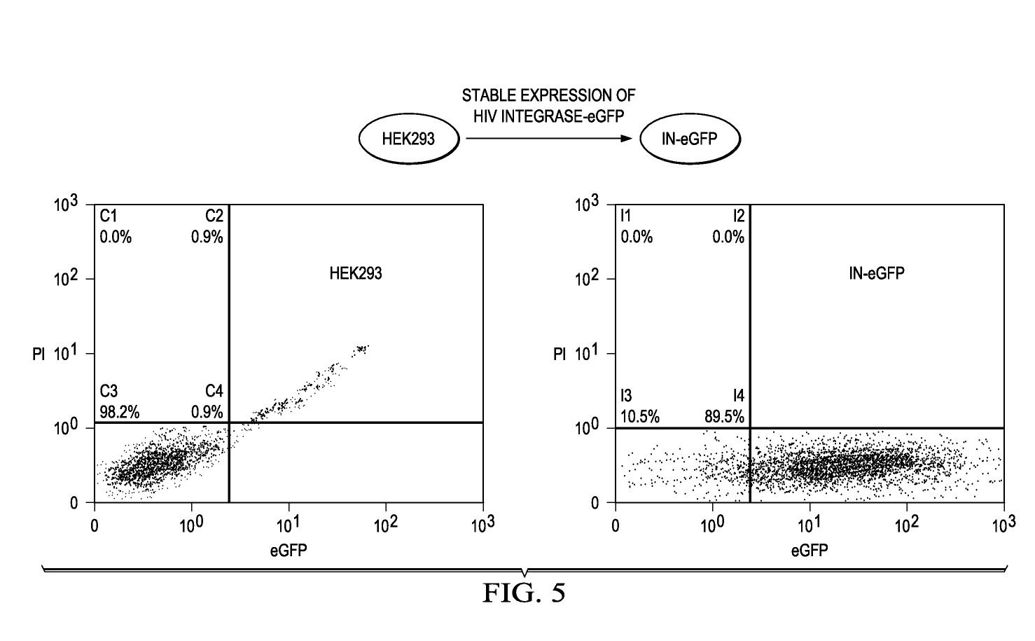

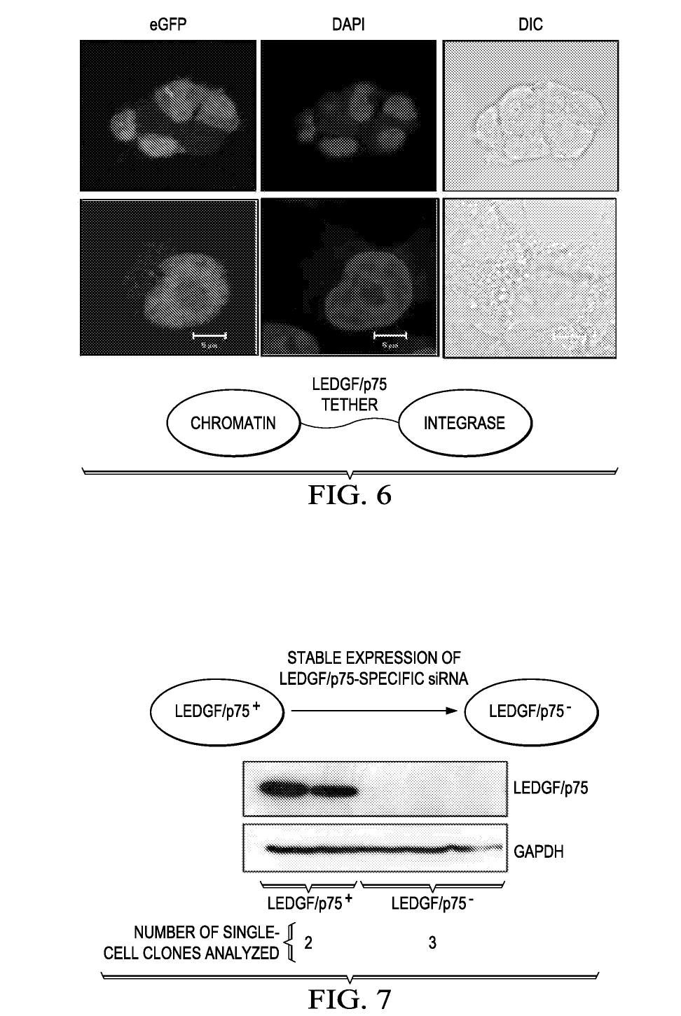

The IN-eGFPc cell line was successfully generated by stable transfection of the pIN-eGFP-IRES-pac expression plasmid and transduction with a lentivirus expressing mCherry. A robust polyclonal cell population was obtained after two weeks of selection in puromycin. High levels of green fluorescence were detected by FACS analysis of these cells (FIG. 5) and, as expected[16], integrase-eGFP was detected in the nuclear compartment by confocal analysis (FIG. 6).

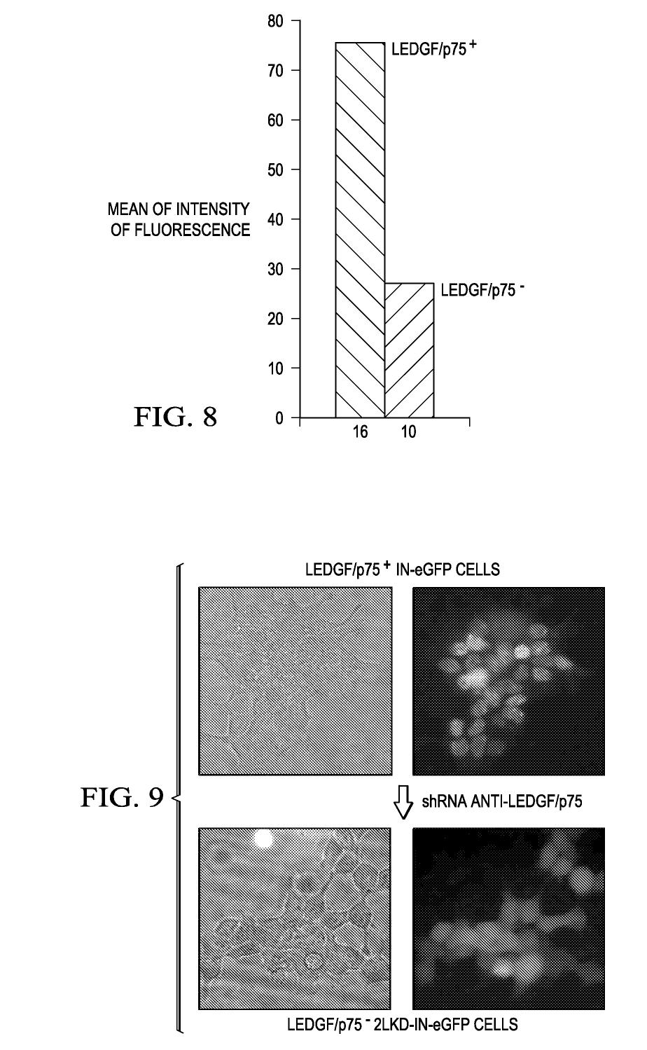

2L_{KD}-IN-eGFP cells were efficiently generated by lentiviral transduction of IN-eGFP cells, several single cell clones expressing high levels of mCherry fluorescence were isolated and analyzed further. LEDGF/p75 levels were undetectable by sensitive immunoblots (FIG. 7) and a decrease in the green fluorescence levels (FIG. 8) as well as a redistribution of the fusion protein was detected (FIGS. 9 and 10A).

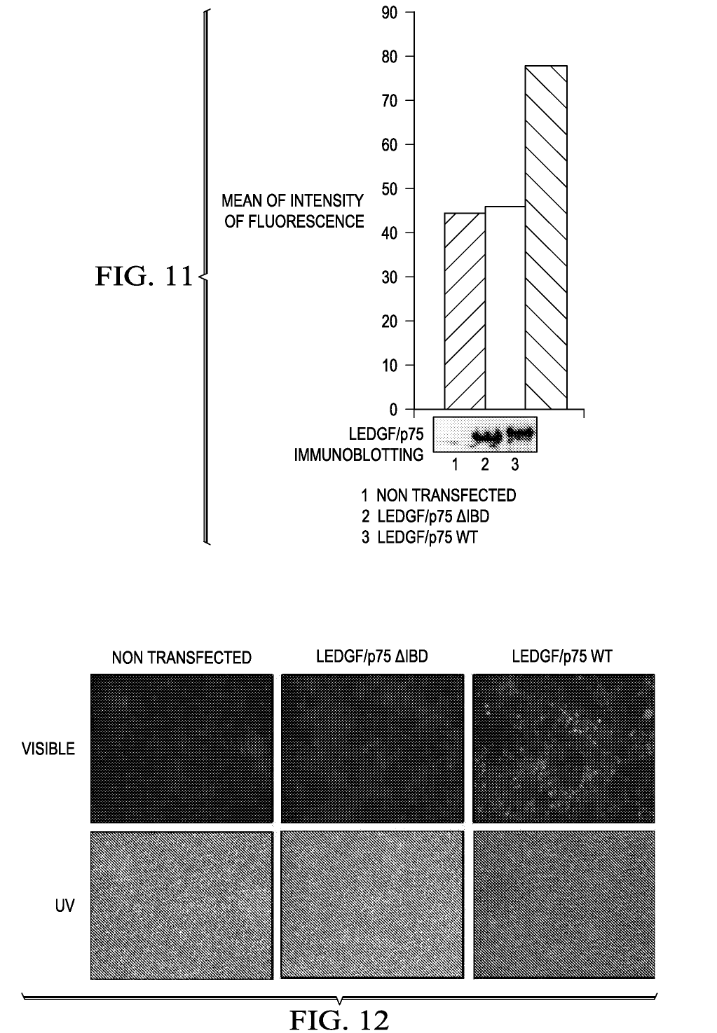

Transient expression of LEDGF/p75 wild type rescued the green fluorescence levels in 2L_{KD}-IN-eGFP cells (FIGS. 11 and 12) and redistribute in-eGFP to the nuclear compartment during cellular interphase and to chromosomes during mitosis (FIG. 10B). However, a LEDGF/p75 mutant lacking the IBD failed to increase the green fluorescence levels of 2L_{KD}-IN-eGFP cells (FIGS. 11 and 12) or to redistribute IN-eGFP to the nuclear compartment or to the chromosomes during mitosis (FIG. 10C).

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

United States Patent Application 20070259358: Integrase cofactor.
WIPO Patent Application No. WO/2008/068765: Compositions and methods for inhibiting HIV-1 replication and integrase activity.
Christ, et al. (2008) Novel treatment strategies in HIV-1 Drug Discovery: Targeting the HIV-1 Integrase-LEDGF/p75 Interaction. 15^{th} Conference on Retroviruses and Opportunistic Infections, Feb. 3-6, 2008, Boston, Mass.
1. Llano M, Saenz D T, Meehan A, Wongthida P, Peretz M, et al. (2006) An essential role for LEDGF/p75 in HIV integration. Science 314: 461-464.
2. Goff S P (2001) Intracellular trafficking of retroviral genomes during the early phase of infection: viral exploitation of cellular pathways. J Gene Med 3: 517-528.
3. Engelman A, Cherepanov P (2008) The lentiviral integrase binding protein LEDGF/p75 and HIV-1 replication. PLoS Pathog 4: e1000046.
4. Poeschla E M (2008) Integrase, LEDGF/p75 and HIV replication. Cell Mol Life Sci 65: 1403-1424.
5. Ciuffi A, Llano M, Poeschla E, Hoffmann C, Leipzig J, et al. (2005) A role for LEDGF/p75 in targeting HIV DNA integration. Nat Med 11: 1287-1289.
6. Shun M C, Raghavendra N K, Vandegraaff N, Daigle J E, Hughes S, et al. (2007) LEDGF/p75 functions downstream from preintegration complex formation to effect gene-specific HIV-1 integration. Genes Dev 21: 1767-1778.
7. Llano M, Vanegas M, Hutchins N, Thompson D, Delgado S, et al. (2006) Identification and characterization of the chromatin-binding domains of the HIV-1 integrase interactor LEDGF/p75. J Mol Biol 360: 760-773.
8. Turlure F, Maertens G, Rahman S, Cherepanov P, Engelman A (2006) A tripartite DNA-binding element, comprised of the nuclear localization signal and two AT-hook motifs, mediates the association of LEDGF/p75 with chromatin in vivo. Nucleic Acids Res 34: 1653-1675.
9. Cherepanov P, Devroe E, Silver P A, Engelman A (2004) Identification of an evolutionarily conserved domain in human lens epithelium-derived growth factor/transcriptional co-activator p75 (LEDGF/p75) that binds HIV-1 integrase. J Biol Chem 279: 48883-48892.
10. Maertens G N, Cherepanov P, Engelman A (2006) Transcriptional co-activator p75 binds and tethers the Myc-interacting protein JPO2 to chromatin. J Cell Sci 119: 2563-2571.
11. Vanegas M, Llano M, Delgado S, Thompson D, Peretz M, et al. (2005) Identification of the LEDGF/p75 HIV-1 integrase-interaction domain and NLS reveals NLS-independent chromatin tethering. J Cell Sci 118: 1733-1743.
12. Yokoyama A, Cleary M L (2008) Menin critically links MLL proteins with LEDGF on cancer-associated target genes. Cancer Cell 14: 36-46.
13. Llano M, Delgado S, Vanegas M, Poeschla E M (2004) Lens epithelium-derived growth factor/p75 prevents proteasomal degradation of HIV-1 integrase. J Biol Chem 279: 55570-55577.
14. Sutherland H G, Newton K, Brownstein D G, Holmes M C, Kress C, et al. (2006) Disruption of Ledgf/Psip1 results in perinatal mortality and homeotic skeletal transformations. Mol Cell Biol 26: 7201-7210.
15. Cherepanov P, Sun Z Y, Rahman S, Maertens G, Wagner G, et al. (2005) Solution structure of the HIV-1 integrase-binding domain in LEDGF/p75. Nat Struct Mol Biol 12: 526-532.
16. Llano M, Vanegas M, Fregoso O, Saenz D, Chung S, et al. (2004) LEDGF/p75 determines cellular trafficking of diverse lentiviral but not murine oncoretroviral integrase proteins and is a component of functional lentiviral preintegration complexes. J Virol 78: 9524-9537.

What is claimed is:

1. A method for screening one or more small molecule inhibitors of HIV-1 integrase comprising the steps of:
providing one or more engineered cells expressing the HIV-1 integrase, wherein the expressed HIV-1 integrase comprises an attached green fluorescent protein;
measuring a baseline green fluorescence signal emanating from the green fluorescent protein attached to the HIV-1 integrase;
adding the one or more small molecule inhibitors dissolved or dispersed in an aqueous or an organic solvent to the one or more engineered cells; and,
measuring a second test green fluorescence signal resulting from the interaction of the one or more small molecule inhibitors and the expressed HIV-1 integrase.

2. The method of claim 1, further comprising the step of measuring a cherry fluorescence signal at periodic intervals, wherein the cherry fluorescent signal emanates from a cherry fluorescent protein contained in the one or more engineered cells and the cherry fluorescent signal is indicative of a health of the one or more engineered cells.

3. The method of claim 2, wherein a decrease in the intensity of the cherry fluorescent signal over time indicates deteriorating health of the one or more engineered cells or the cell line or the cell culture.

4. The method of claim 1, wherein the one or more engineered cells comprises at least two fluorescent proteins.

5. The method of claim 1, wherein the one or more engineered cells expresses at least one fluorescent protein attached to the expressed HIV-1 integrase.

6. The method of claim 1, wherein the one or more engineered cells expresses at least one fluorescent protein that is not attached to the HIV-1 integrase.

7. The method of claim 1, wherein the one or more small molecule inhibitors are selected from a group consisting of organic compounds, heterocyclic aromatic and acyclic heteroatom compounds, dioxybutyric acids and derivatives, propanediones and derivativess, naphthyridine-carboxamides and derivatives, naphthalenyl ketones and derivativess, hydroxynaphthyridinone carboxamides and derivatives, hydroxypyrrole derivatives, tricyclic analogs of hydroxy polyhydronaphthyridine dione compounds, and nitrogenous condensed ring compounds.

8. The method of claim 1, wherein the one or more engineered cells are selected from cells, cell lines, or cell cultures selected from a group consisting of human embryonic kidney cells, Chinese hamster ovary (CHO) cells, HeLa cell lines, COS cell lines, mammalian cells, and bacterial cells.

9. The method of claim 1, wherein the organic solvent comprises ketones, alcohols, dimethyl sulfoxide, esters, ethers, acids or any combinations thereof.

10. The method of claim 1, wherein a decrease in intensity of the test green fluorescent signal when compared to the intensity of the baseline green fluorescent signal indicates an inhibition of the one or more enzymes by the one or more small molecule inhibitors.

11. A method for screening one or more small molecule inhibitors of HIV-1 integrase comprising the steps of:
providing one or more engineered human embryonic kidney cells expressing the HIV-1 integrase, wherein the expressed HIV-1 integrase comprises an attached green fluorescent protein;
measuring a baseline green fluorescence signal emanating from the green fluorescent protein attached to the HIV-1 integrase;
adding the one or more small molecule inhibitors dissolved or dispersed in an aqueous or an organic solvent to the one or more engineered human embryonic kidney cells; and
measuring a second test green fluorescence signal resulting from the interaction of the one or more small molecule inhibitors and the expressed HIV-1 integrase.

12. The method of claim 11, further comprising the step of measuring a cherry fluorescence emanating from a cherry fluorescent protein contained in the one or more engineered human embryonic kidney cells at periodic intervals, wherein the cherry fluorescence is indicative of a health of the one or more engineered human embryonic kidney cells.

13. The method of claim 12, wherein a decrease in the intensity of the cherry fluorescent signal over time indicates deteriorating health of the one or more engineered human embryonic kidney cells.

14. The method of claim 11, wherein the one or more small molecule inhibitors are selected from a group consisting of organic compounds, heterocyclic aromatic and acyclic heteroatom compounds, dioxybutyric acids and derivatives, propanediones and derivativess, naphthyridine-carboxamides and derivatives, naphthalenyl ketones and derivativess, hydroxynaphthyridinone carboxamides and derivatives, hydroxypyrrole derivatives, tricyclic analogs of hydroxy polyhydronaphthyridine dione compounds, and nitrogenous condensed ring compounds.

15. The method of claim 11, wherein a decrease in intensity of the test green fluorescent signal when compared to the intensity of the baseline green fluorescent signal indicates an inhibition of the HIV-1 integrase enzyme by the one or more small molecule inhibitors.
